(19) United States
(12) Patent Application Publication
(10) Pub. No.: US 2004/0248984 A1
(43) Pub. Date: Dec. 9, 2004

(54) Use of $g(b)-adrenoceptor agonists for the treatment of neurodegenerative diseases

(76) Inventors: Josef Krieglstein, Colbe (DE); Carsten Culmsee, Gladenbach (DE); Vera Junker, Marburg (DE); Helmut Blum, Dusseldorf (DE); Wolfgang Greb, Dusseldorf (DE)

Correspondence Address: GUDRUN E. HUCKETT DRAUDT, LONSSTR. 53, WUPPERTAL, 42289, DE

(21) Appl. No.: 10/488,500

(22) PCT Filed: Aug. 22, 2002
(86) PCT No.: PCT/EP02/09369

(30) Foreign Application Priority Data
Aug. 29, 2001 (DE) 10142175.3
Aug. 29, 2001 (DE) 10142176.1
Aug. 29, 2001 (DE) 10142178.8

(51) Int. Cl. A61K 31/165; A61K 31/137

(52) U.S. Cl. 514/620; 514/651

(57) ABSTRACT

The use of β-adrenoceptor agonists for restoring and/or maintaining the function of partially or completely damaged/degenerated cells in the central nervous system and/or other nerve cells is claimed. The use of β2-adrenoceptor agonists leads to activation of astrocytes and initiation of endogenous processes of neuroprotection, it thus being possible for the damage or destruction of nerve cells to be reduced and, in some cases, even prevented.

The present invention relates to the use of β-adrenoceptor agonists for the treatment of neurodegenerative diseases.

The human brain is a highly complicated organ with more than 100 billion nerve cells (=neurons) and about 10 000 connections (=synapses) per cell. The brain is the central organ of the conscious and unconscious processing of the stimuli acting on the human body, of thought and feeling, of deliberate action, of learning and of memory. One of the most important functions of the human brain is information processing in speech; also control center for a large number of organ functions, and of breathing, of the heart rate and of temperature regulation.

A large number of diseases lead to the death of nerve cells and/or to a reduction in synapses and thus to a restriction of brain function. Examples of such pathological states are Alzheimer's disease, cerebrovascular dementias, Parkinson's disease, Pick's disease, Huntington's chorea, amyotrophic lateral sclerosis, Lewy body dementia, stroke and brain trauma such as cerebral contusion and concussion, and injuries to the brain and spinal cord or transverse lesions, spina bifida, and diseases of the inner ear, for example diseases associated with the occurrence of tinnitus, such as subacute or chronic tinitus, sudden loss of hearing, Menière's disease, and diseases associated with a restriction of audition or with the reduction in vision etc. There is as yet no clinically established neuroprotective therapy of said pathological states. Only symptoms, but not the causes thereof, are treated.

The aim of causal therapy is to prevent the death of nerve cells.

There is at present no established therapy with which it is possible for nerve cells to be protected from damage or regenerated. An essential element of the therapy applied at present to the above-mentioned disorders is to prevent indirect or secondary damage to nerve cells, such as, for example, to increase cerebral blood flow or restore it if a vessel is occluded. However, this type of therapy is successful only, if at all, when it can be employed rapidly after the acute event.

The present invention was based on the object of finding drug substances able to protect nerve cells from damage and to restore at least in part the function of partially or completely degenerated cells.

It has surprisingly been found that activation of astrocytes with drug substances such as β-adrenoceptor agonists initiates endogenous processes of neuroprotection, thus making it possible to reduce, and in some cases even to prevent, damage or destruction of nerve cells.

The present invention accordingly relates to the use of β-adrenergic agonists for restoring and/or maintaining the function of partially or completely damaged cells in the central nervous system and/or other nerve cells.

For the purposes of the present invention, “damaged cell” means that the cell has been damaged by external effects or is partially or completely destroyed in the sense of degeneration through processes taking place in the cell, which may be associated with an impairment of body functions. The term “cell damage” includes both damage to individual cells or cell types and damage to strings or tracts of nerve cells.

The nerve cells include, besides the cells of the central nervous system, also the cells of the spinal cord and all other nerve cells present in the body.

β-Adrenoceptors respond in particular to adrenergic drug substances. Examples of β-adrenergic agonists which are preferably employed in the present invention because of their good activity are clenbuterol, formoterol, fenoterol, salbutamol, orciprenaline, isoetharine, cimaterol, ractopamine, reproterol, salmeterol, terbutaline, their isomers, acid addition salts, analogs and any mixtures of the foregoing.

The aforementioned β-adrenergic agonists are in some cases known drug substances. Thus, for example, clenbuterol is known from the prior art as an agent for astma.

It has been possible to demonstrate by means of experiments for example that the lipophilic β-adrenoceptor agonists are able to permeate into the brain and there stimulate the β-adrenoceptors of the astrocytes. Stimulation of these receptors in turn leads to an activation of the astrocytes and consequently to an increased release of growth factors, such as NGF, which are able to protect nerve cells from damage.

The β-adrenoceptor agonists are administered for the therapy in the amounts customary for these pharmaceuticals, in particular in an amount of from 0.01 to 100 mg/day, it also being possible for preferred ranges of amounts to depend on the particular β-adrenoceptor agonist. With substances such as clenbuterol, formoterol, fenoterol and salmeterol, a particularly good neuroprotective effect is obtained when they are administered in an amount of from 0.01 to 5 mg/day. Terbutaline is administered preferably in an amount of from 1.0 to 30 mg/day, salbutamol in an amount of from 1.0 to 50 mg/day, and orciprenaline and reproterol in an amount of from 1.0 to 100 mg/day.

It is also possible for β1-adrenoceptor agonists such as dobutamine to activate astrocytes and thus achieve protection of neurons. In one possible embodiment, the β-adrenoceptor agonists used according to the invention are employed in combination with the β1- and/or β2-adrenoceptor agonists.

In a further possible embodiment of the present invention, the β-adrenergic agonists are employed in combination with NMDA antagonists, thus applying a supplementary or further principle of action.

NMDA antagonists, such as, for example, the adamantane derivatives, are known compounds which are frequently also employed for the treatment of various diseases. Thus, for example, the dopaminergic effect of amantadine (1-adamantanamine) is known.

European patent application EP 392 059 describes the use of adamantane derivatives for the prevention and treatment of cerebral ischemia. According to this publication, the destruction of brain cells following an ischemia is protectively prevented through the use of the adamantane derivatives in that the adamantane derivatives are employed as antagonists for the NMDA receptor channels of the nerve cells in the brain.

Adamantane derivatives having formula I are preferably employed

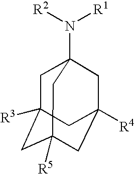

in which

R¹ and R² are identical or different and are hydrogen or a straight-chain or branched C₁-C₆-alkyl group, or together with the N atom may represent a heterocyclic group having 5 or 6 ring atoms,

R³ and R⁴ are identical or different and are hydrogen, a straight-chain or branched C₁-C₆-alkyl group or a C₅-C₆-cycloalkyl group or a vinyl group, and

R⁵ is hydrogen or a straight-chain or branched C₁-C₆-alkyl group.

The adamantane derivatives having formula I can be employed in the form of their compounds described by formula I or in the form of their pharmaceutically acceptable salts. Pharmaceutically acceptable salts which can preferably be employed include the acid addition salts such as the hydrochlorides, hydrobromides, sulfates, acetates, succinates, tartrates, with preference for the hydrochlorides.

Preferred compounds having formula I are those in which R¹, R² and R⁴ are hydrogen and R and R⁵ are a methyl and/or ethyl group.

In a particularly preferred compound, R¹, R² and R⁴ are hydrogen and R³ and R⁵ are a methyl radical, or the hydrochloride thereof. This compound is known under the INN memantine.

The β-adrenoceptor agonists used according to the invention and, where appropriate, further customary drug substances which do not adversely affect the therapy, or which support it, and conventional ingredients, can be present in pharmaceutically customary dosage forms, in particular as solution, suspension, emulsion, tablets, suppository, etc. Use in special formulations such as liposomes, nanosomes, slow-release pellets etc. is also possible. They can be administered in a conventional way, for example orally, parenterally, intravenously, by inhalation, nasally, rectally, intraventricularly, intraarterially, intraperitoneally and/or intramuscularly or as an implant. The mode of administration is preferably selected so that the impaired cells can be reached by the drug substance of the invention in the fastest possible manner.

The β-adrenoceptor agonists used according to the invention are particularly suitable for producing medicaments for the treatment of neurodegenerative diseases. Examples of such diseases are Alzheimer's disease, cerebrovascular dementias, Parkinson's disease, Pick's disease, Huntington's chorea, amyotrophic lateral sclerosis, Lewy body dementia, stroke and brain trauma such as cerebral contusion and concussion, and injuries to the brain and spinal cord or transverse lesions, spina bifida, and diseases of the inner ear, for example diseases associated with the occurrence of tinnitus, such as subacute or chronic tinitus, sudden loss of hearing, Menière's disease, and diseases associated with a restriction of audition or with the reduction in vision etc.

In a further embodiment of the present invention, the β-adrenergic agonists are employed to restore and/or maintain the function of cells of the central nervous system which have been partially or completely damaged by encephalopathy and/or of other nerve cells. Encephalopathy is one of the pathological non-inflammatory changes in the brain with variable neurological and/or mental symptoms. Examples of encephalopathies which can be treated according to the invention with β-adrenergic agonists are toxic encephalopathy, diabetic encephalopathy, hepatic encephalopathy, hypertensive encephalopathy, metabolic encephalopathy, such as encephalopathy caused by metabolic disturbances, e.g. associated with enzymopathies, endogenous disturbances, renal failure (uremic encephalopathy), liver diseases, disturbances of the water/electrolyte or acid/base balance, myoclonic infantile encephalopathy (Kinsboorne syndrome), infantile postictereca encephalopathy (bilirubin encephalopathy), postcombustional encephalopathy, encephalopathy caused by heavy metals, in particular by inorganic and organic heavy metal compounds such as compounds of lead, mercury, and amalgam, thallium, bismuth, aluminum, nickel and any mixtures of these compounds and their metal alloys, toxic encephalopathy caused by alcohol, bovine spongiform encephalopathy (BSE), supcortical progressive encephalopathy, traumatic encephalopathy.

In a further embodiment of the present invention, the compounds used according to the invention are employed to prevent the aforementioned diseases.

In a further embodiment of the present invention, the compounds used according to the invention are employed as additive(s) for culture media to promote growth and/or differentiation and/or protection of mammalian cells and human cells.

### EXAMPLES

1) Activation of Astrocytes

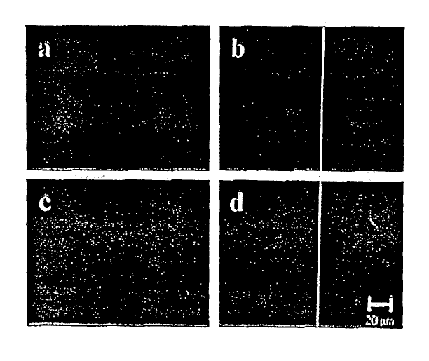

Primary cultures of astrocytes were obtained from the cerebral cortical tissue of newborn Fischer 344 rats within 24 hours after birth. The brains were dissected out of the vault of the cranium under sterile conditions, the cortical tissue (cortex) was isolated, and the cells were dissociated through a narrow-mesh wire netting. The cells were put into cell culture bottles and cultivated in serum-containing DMEM solution (contained fetal calf serum and a penicillin/streptomycin mixture) until the cells were confluent. Oligodendrocytes and microglia were removed by washing with cold buffer solution. The confluent astrocytes were then detached from the base of the culture bottles with a trypsin solution and seeded in a density of 20 000 cells/cm2 in Petri dishes on coverslips and cultivated in serum-containing medium until the cells were again confluent. Two days after confluence, the medium was replaced by serum-free medium and, after 24 hours, the medium was replaced again, likewise by serum-free medium. Twenty-four hours after the second replacement of medium, salmeterol was added. Six hours after the treatment, the astrocytes were photographed under the microscope with 200× magnification to document the morphological changes (FIG. 1/7). The figure shows the astrocytes 6 hours after the start of the treatment. The changes from the polygonal, flat cells of low refractility in the controls to the activated, stellate and refractile astrocytes in the salmeterol-treated groups are clearly evident.

2) Induction of NGF in Cultivated Rat Hippocampal Neurons

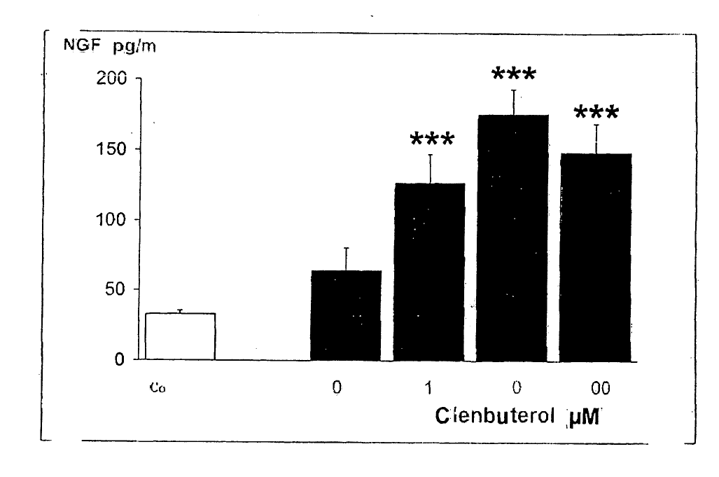

Primary mixed cultures with a proportion of 50% each of neurons and astrocytes were obtained from the hippocampus of newborn Fischer 344 rats within 24 hours after birth. The hippocampus was isolated from the rats' brains under sterile conditions and, after brief incubation in a papain solution, cautiously triturated using a glass pipette. The cells dissociated in this way were seeded in a density of 3×10⁵ cells in 35 mm Petri dishes and cultivated in serum-containing medium (MEM with 10% NU-Serum and a penicillin/streptomycin mixture). Two days after cultivation, cytosine arabinofuranoside was put in the medium for 24 hours in order to inhibit the growth of the astrocytes. Every 3-4 days, the medium was replaced by fresh serum-containing medium. After 14 days in culture, the medium was replaced by serum-free medium and, after a further 24 hours, clenbuterol was added. Four hours after the start of incubation with clenbuterol, the culture medium was collected. The NGF content in the medium was determined by means of a standardized enzyme-coupled immune reaction (ELISA). For this purpose, the chambers of a multiwell plate were coated with an NGF antibody and then incubated in the individual chambers with the medium of the various groups. The NGF bound from the medium in the chambers in this way was then incubated with a beta-galactosidase-conjugated NGF antibody. This was followed by beta-galactosidase-catalyzed conversion of chlorophenol red beta-galactopyranoside into a red dye which was measured by photometry. To construct a standard plot, standard dilutions of NGF were measured in addition to the samples. The NGF content in the samples was determined from the standard plot (FIG. 2/7).

3) Neuroprotective Effect of Clenbuterol In Vitro

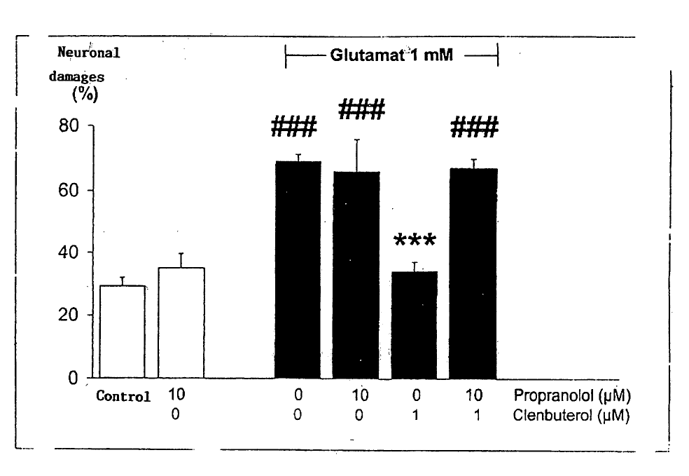

Primary mixed cultures from the rat hippocampus were set up as described in Example 2 and, after 14 days in culture, subjected to a medium change to serum-free medium. Twenty-four hours after the medium change, the adrenoceptor antagonist propranolol was put into the medium. A further 20 minutes later, the β₂-adrenoceptor agonist clenbuterol was added, and the cells were incubated thus for 4 hours. Sister cultures of the hippocampal cells treated in this way received only vehicle, propranolol or clenbuterol alone. After 4 hours, the medium was replaced and the cells were incubated in serum-free medium with L-glutamate (1 mM) for 1 hour. The medium was then again replaced by serum-free medium in order to remove the glutamate from the cultures. Propranolol and clenbuterol were added fresh again at each medium replacement and were thus present in the medium during the glutamate treatment and up to 18 hours thereafter. Eighteen hours after the glutamate damaging, the cells were incubated with a trypan blue solution and fixed, and the damaged, blue-stained neurons were quantified under the microscope at a magnification of 200× (FIG. 3/7).

4) Cerebroprotective Effect of Clenbuterol in a Rat Cerebral Ischemia Model

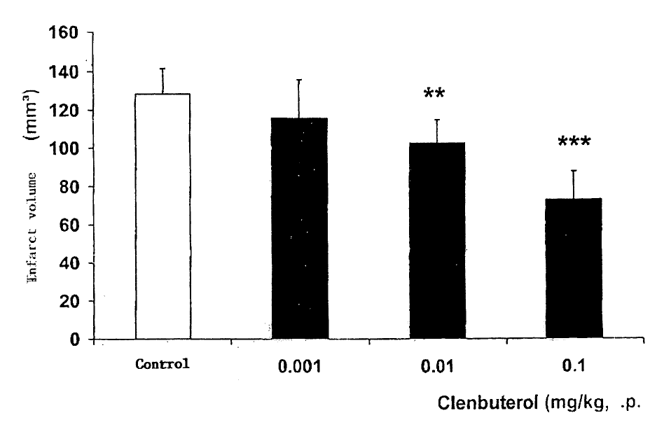

An ischemia was induced in the cortical tissue (cortex) of male Long Evans rats by permanent occlusion of the middle cerebral artery (arteria cerebri media, MCA). The surgical procedure took place under inhalation anesthesia (1.5% halothane in a 30:70 oxygen/nitrous oxide mixture). Under deep anesthesia, the left temporal muscle was incised between the eye and ear, and the cranium exposed in this way was trepanned under stereomicroscopic control. The dura mater was removed, and the MCA was sclerosed at three places by means of bipolar electrocoagulation. The wound in the temporal muscle was then closed in order to retain the function of the muscle for food intake. The body temperature of the rats was controlled at 37±0.5° C. by a heated underlay during the operation and kept stable for a further 2 hours after the operation with the aid of a heating lamp at an ambient temperature of 30° C. During the operation, moreover, physiological parameters (blood pressure, plasma glucose level, arterial pH, CO₂ and O₂ partial pressures) were monitored and recorded. Seven days after closure of the MCA, the brains were removed and frozen. A kryomicrotome was used to prepare coronal sections (20 μM) of the brains at defined distances of 0.5 mm. The brain sections were then stained with cresyl violet, whereupon the infarct region showed only a slight coloration and could thus be distinguished from the healthy tissue. The infarct area of the individual sections was measured and the infarct volume was calculated from the values for the areas and the defined distance of the consecutive sections. Clenbuterol was administered intraperitoneally in the various doses 3 hours before occlusion of the MCA (FIG. 4/7).

5) Neuroprotective Effect of Salmeterol In Vitro

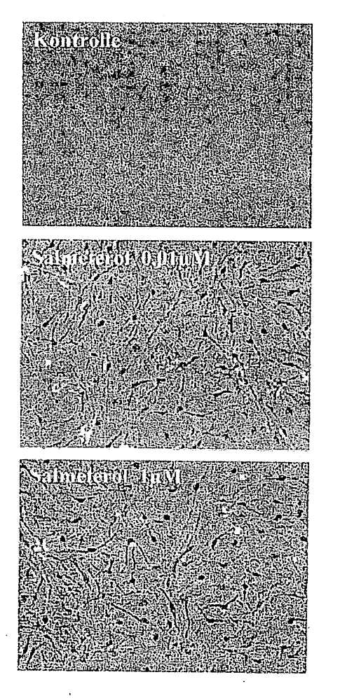

Primary mixed cultures from the rat hippocampus were set up as described in Example 2 and, after 14 days in culture, subjected to a medium change to serum-free medium. Twenty-four hours after the medium change, the β-adrenoceptor agonist salmeterol was added and the cells were incubated thus for 4 hours. Sister cultures of the hippocampal cells treated in this way received only vehicle, or salmeterol alone. After 4 hours, the medium was replaced and the cells were incubated in serum-free medium with L-glutamate (1 mM) for 1 hour. The medium was then again replaced by serum-free medium in order to remove the glutamate from the cultures. Salmeterol was added fresh again at each medium replacement and was thus present in the medium during the glutamate treatment and up to 18 hours thereafter. Eighteen hours after the glutamate damaging, the cells were incubated with a trypan blue solution and fixed, and the damaged, blue-stained neurons were quantified under the microscope at a magnification of 200× (FIG. 5/7). The stated values are means and standard deviation from 5-6 cultures per group. *p<0.05; **p<0.01; and ***p<0.001 compared with the glutamate-treated control (analysis of variance, Scheffe test).

6) Neuroprotective Effect of Salmeterol In Vivo

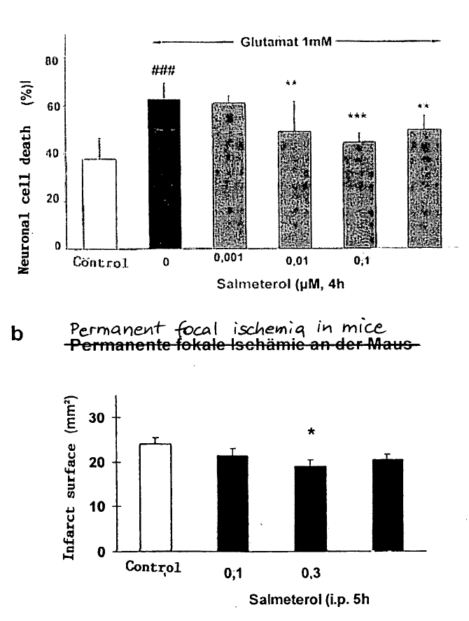

A focal cerebral ischemia was produced in mice by ligating the middle cerebral artery. Male NMRI mice (26-31 g, 10-12 animals per group) were used for the experiments. The animals were anesthetized by an intraperitoneal injection of tribromoethanol (600 mg/kg). The surgical field was then opened by a 2 cm-long incision between the left eye and ear, the temporal muscle was removed by thermocautery, and a fine drill was used to remove the bone in order to expose the middle cerebral artery. This artery and its two distal branches were permanently occluded. During the dissection, the body temperature of the mouse was measured and kept constant at 37+/−1° C. by an infrared heating lamp. After the dissection, the animals were left at an ambient temperature of 30° C. for a further two hours. To determine the infarcted region, the mice were again anesthetized with tribromoethanol 48 hours after the occlusion of the middle cerebral artery and perfused with a 1.5% strength neutral red solution (0.5 ml intraperitoneally). This revealed the perfused brain tissue as red and the infarcted region remained pale. The isolated brains were fixed with a 4% formaldehyde buffer (pH 7.4) for at least 24 hours and then the unstained region on the surface of the brain (infarct region) was measured with computer assistance (NIH image software). The salmeterol dissolved in 0.9% NaCl was injected interperitoneally 5 hours before the operation. The animals in the control group received only 0.9% NaCl solution (FIG. 6/7). The values are means and standard deviation of 15-16 animals per group. *p<0.05 compared with the control (analysis of variance, Duncan's test).

7) Neuroprotective Effect of Clenbuterol and Memantine In Vivo

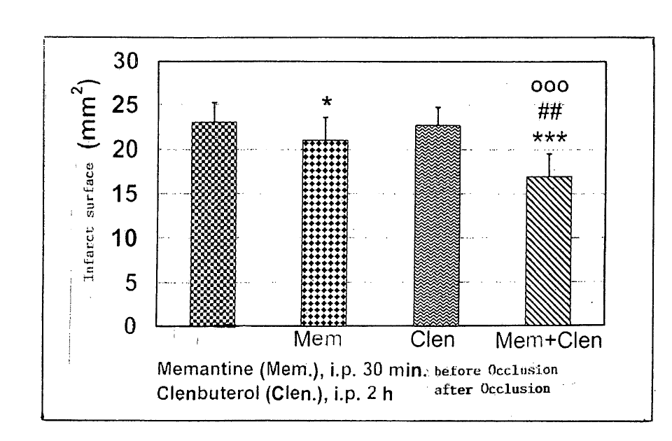

A focal cerebral ischemia was produced in mice by ligating the middle cerebral artery. Male NMRI mice (26-31 g, 10-12 animals per group) were used for the experiments. The animals were anesthetized by an intraperitoneal injection of tribromoethanol (600 mg/kg). The surgical field was then opened by a 2 cm-long incision between the left eye and ear, the temporal muscle was removed by thermocautery, and a fine drill was used to remove the bone in order to expose the middle cerebral artery. This artery and its two distal branches were permanently occluded. During the dissection, the body temperature of the mouse was measured and kept constant at 37+/−1° C. by an infrared heating lamp. After the dissection, the animals were left at an ambient temperature of 30° C. for a further two hours. To determine the infarcted region, the mice were again anesthetized with tribromoethanol 48 hours after the occlusion of the middle cerebral artery and perfused with a 1.5% strength neutral red solution (0.5 ml intraperitoneally). This revealed the perfused brain tissue as red and the infarcted region remained pale. The isolated brains were fixed with a 4% formaldehyde buffer (pH 7.4) for at least 24 hours and then the unstained region on the surface of the brain (infarct region) was measured with computer assistance (NIH image software). The two drugs to be investigated, memantine and clenbuterol, were dissolved in 0.9% NaCl for the injection. Memantine (20 mg/kg) was injected interperitoneally 30 minutes before the operation and clenbuterol 2 hours after the operation. The animals in the control group received only 0.9% NaCl solution (FIG. 7/7).

1. The use of β-adrenoceptor agonists for restoring and/or maintaining the function of partially or completely damaged cells of the central nervous system and/or other nerve cells.

2. The use as claimed in claim 1, characterized in that astrocytes and/or endogenous protective mechanisms are activated or stimulated.

3. The use as claimed in claim 1, characterized in that the β-adrenergic agonists are selected from clenbuterol, formoterol, fenoterol, salbutamol, orciprenaline, isoetharine, cimaterol, ractopamine, reproterol, salmeterol, terbutaline, their isomers, acid addition salts, analogs and any mixtures of the foregoing.

4. The use as claimed in claim 1, characterized in that the β-adrenoceptor agonists in an amount of from 0.01 to 100 mg/day.

5. The use as claimed in claim 4, characterized in that substances such as clenbuterol, formoterol, fenoterol, and salmeterol are administered in an amount of from 0.01 to 5 mg/day, terbutaline in an amount of from 1.0 to 30 mg/day, salbutamol in an amount of from 1.0 to 50 mg/day and orciprenaline and reproterol in an amount of from 1.0 to 100 mg/day.

6. The use as claimed in claim 1, characterized in that β1-adrenoceptor agonists such as dobutamine are used.

7. The use as claimed in claim 1, characterized in that NMDA antagonists are used.

8. The use as claimed in claim 1, characterized in that the neurodegenerative diseases are selected from Alzheimer's disease, cerebrovascular dementias, Parkinson's disease, Pick's disease, Huntington's chorea, amyotrophic lateral sclerosis, Lewy body dementia, stroke and/or brain trauma such as cerebral contusion and concussion, and injuries to the brain and spinal cord or transverse lesions, spina bifida, and diseases of the inner ear, for example diseases associated with the occurrence of tinnitus, such as subacute or chronic tinitus, sudden loss of hearing, Menière's disease, and diseases associated with a restriction of audition or with the reduction in vision etc.

9. The use as claimed in claim 1, characterized in that the neurodegenerative diseases are selected from toxic encephalopathy, diabetic encephalopathy, hepatic encephalopathy, hypertensive encephalopathy, metabolic encephalopathy, such as encephalopathy caused by metabolic disturbances, e.g. associated with enzymopathies, endogenous disturbances, renal failure (uremic encephalopathy), liver diseases, disturbances of the water/electrolyte or acid/base balance, myoclonic infantile encephalopathy (Kinsboorne syndrome), infantile postictereca encephalopathy (bilirubin encephalopathy), postcombustional encephalopathy, encephalopathy caused by heavy metals, in particular by inorganic and organic heavy metal compounds such as compounds of lead, mercury, and amalgam, thallium, bismuth, aluminum, nickel and any mixtures of these compounds and the metal alloys, toxic encephalopathy caused by alcohol, bovine spongiform encephalopathy (BSE), supcortical progressive encephalopathy, traumatic encephalopathy.

10. The use as claimed in claim 1, characterized in that the compounds are employed for preventing neurodegenerative diseases.

11. The use as claimed in claim 1 as additive for culture media to promote growth and/or differentiation and/or protection of mammalian cells and human cells.
